(19) United States
(12) Patent Application Publication
(10) Pub. No.: US 2013/0089930 A1
(43) Pub. Date: Apr. 11, 2013

(54) MICRODEVICE FOR FUSING CELLS

(71) Applicants: Sang-Woo Joo, Gyeongsan-si (KR); Shizhi Qian, Gyeongsan-si (KR); Ning Hu, Gyeongsan-si (KR)

(72) Inventors: Sang-Woo Joo, Gyeongsan-si (KR); Shizhi Qian, Gyeongsan-si (KR); Ning Hu, Gyeongsan-si (KR)

(21) Appl. No.: 13/282,856

(22) Filed: Oct. 27, 2011

(30) Foreign Application Priority Data
Oct. 6, 2011 (KR) 10-2011-0101882

(51) Int. Cl. C12N 15/02; C12M 1/42

(52) U.S. Cl. 435/450; 435/285.2

(57) ABSTRACT

A microdevice for fusing cells including: a microchannel layer including a main microchannel and a plurality of sub-microchannels branched from one end of the main microchannel; a plurality of first electrodes formed on one side of the main microchannel; a plurality of second electrodes formed on the other side of the main microchannel and each second electrode facing the each of the first electrodes; a thin film disposed on the microchannel layer and covering the main microchannel; an upper cover including an air inflow passage for connecting a top of the thin film and the outside of the microdevice; and a power supply unit for applying voltage to the plurality of first and second electrodes.

### CROSS-REFERENCE TO RELATED PATENT APPLICATION

This application claims the benefit of Korean Patent Application No. 10-2011-0101882, Oct. 6, 2011, in the Korean Intellectual Property Office, the disclosure of which is incorporated herein in its entirety by reference.

### BACKGROUND OF THE INVENTION

1. Field of the Invention

The present invention relates to a microdevice for fusing cells for electrofusion, which manufactures a desired fused cell at high efficiency.

2. Description of the Related Art

Cell fusion is a method of preparing a hybrid cell by artificially fusing two cells in different types. The cell fusion may be performed by using chemicals or an electric pulse. Here, combining two cells in different types by porating a cell membrane via an electric pulse is referred to as electrofusion.

There are mainly four continuous phases in the electrofusion: dielectrophoresis-based cell alignment, reversible electroporation, reconstruction of cytomembrane, and karyon fusion. Generally, the dielectrophoresis-based cell alignment needs a sinusoidal alternating current (AC) electric field (intensity: 100 to 300 V/cm) to exert a positive dielectrophoretic (DEP) force on the cells. In addition, a high-strength DC electric pulse signal series is required in the reversible electroporation (intensity: 1 to 10 kV/cm, pulse width: 10 to 50 μs).

Plate electrodes are usually used in a conventional cell electrofusion device. In general, a distance between two plate electrodes is equal to or above 1 cm, and as a result, an expensive generator is required to obtain high-strength electric pluses. Moreover, an electric field generated between the plate electrodes is uniform, and thus probabilities of occurrence of reversible electroporation and electrofusion of aligned cells are equal. Thus, a probability of occurrence of unwanted multi-cell electrofusion in the conventional cell electrofusion device is relatively high.

In order to increase pairing precision, fusion efficiency, multi-function integration, and a degree of automation, a micro electromechanical system (MEMS) and microfluidic technology have been used to develop microchips for electrofusion. Microstructures in these microchips have a similar scale as cells (5 to 50 μm), and thus useful in more precise cell manipulation. Also, owing to a short distance between two microelectrodes, a high electric field required for cell fusion may be generated even with a low voltage, and thus difficulties of power supply and high manufacturing costs may be reduced.

However, in a conventional microfluidic device, an average cell fusion efficiency is about 40%, which is higher than a general chemical fusing method (use polyethylene glycol (PEG), less than 5%) and a conventional electrofusion method (less than or equal to 12%), but a probability of forming desired cell-cell twins is only from 42 to 68%. Accordingly, fusion efficiency of total cells is about 40%×42-68%, i.e., 16 to 30%. In other words, when a cell A and a cell B are to be fused, undesired hybrid products, such as AA, ABB, AABB, AAB, and BB, may be excessively obtained instead of AB.

Accordingly, a new microfluidic chip for fusing desired cells at higher efficiency is required to be developed.

### SUMMARY OF THE INVENTION

The present invention provides a microdevice for fusing cells, wherein cells to be fused are effectively fused in a one-to-one manner.

According to an aspect of the present invention, there is provided a microdevice for fusing cells, the microdevice including: a microchannel layer including a main microchannel and a plurality of sub-microchannels branched from one end of the main microchannel, wherein an outlet hole is formed at the other end of the main microchannel and a first cell inlet hole and a second cell inlet hole are respectively formed at ends of each of the plurality of sub-microchannels; a plurality of first electrodes formed on one side of the main microchannel; a plurality of second electrodes formed on the other side of the main microchannel and each second electrode facing the each of the first electrodes; a thin film disposed on the microchannel layer and covering the main microchannel; an upper cover including an air inflow passage for connecting a top of the thin film and the outside of the microdevice; and a power supply unit for applying voltage to the plurality of first electrodes and the plurality of second electrodes.

According to another aspect of the present invention, there is provided a method of fusing cells, the method including: providing the microdevice; bending a thin film toward a main microchannel covered by the thin film by injecting air to a top of the thin film through an air inflow passage of a top cover; injecting first cells and second cells into respective inlet holes, and flowing the first and second cells through a sub-microchannel to the main microchannel; applying an alternating current (AC) voltage between a first electrode and a second electrode such that the injected first and second cells are aligned in the main microchannel according to a dielectrophoresis; performing electroporation on the aligned first and second cells by applying direct current (DC) pulses between the first electrode and the second electrode; applying a quasi-damping AC voltage between the first electrode and the second electrode such that the electroporated first and second cells are fused by being adjacently disposed to each other according to a dielectrophoresis; relaxing the deformed thin film by releasing the air; and obtaining the fused first and second cells through an outlet hole.

### BRIEF DESCRIPTION OF THE DRAWINGS

The above and other features and advantages of the present invention will become more apparent by describing in detail exemplary embodiments thereof with reference to the attached drawings in which:

FIG. 1 is a perspective view of a microdevice for fusing cells, according to an embodiment of the present invention;

FIG. 2 is an exploded perspective view of a microdevice for fusing cells, according to an embodiment of the present invention;

FIG. 3 is an exploded perspective view of a lower portion, a thin film, and an upper cover of a microdevice for fusing cells, according to an embodiment of the present invention;

FIG. 4 is a perspective view of the lower portion according to an embodiment of the present invention;

FIG. 5 is a perspective view of a substrate according to an embodiment of the present invention;

FIG. 6 is a perspective view of a microchannel layer according to an embodiment of the present invention;

FIG. 7 is a perspective view of a structure of an electrode according to an embodiment of the present invention;

FIG. 8 is a perspective view of a structure of a thin film according to an embodiment of the present invention;

FIG. 9 is a perspective view of a structure of the upper cover according to an embodiment of the present invention; and

FIGS. 10A through 10F are schematic internal cross-sectional views for describing operations of a microdevice for fusing cells.

### DETAILED DESCRIPTION OF THE INVENTION

Hereinafter, embodiments of the present invention will be described in detail. The invention may, however, be embodied in many different forms and should not be construed as being limited to the embodiments set forth herein; rather, these embodiments are provided so that this disclosure will be thorough and complete, and will fully convey the concept of the invention to those skilled in the art.

The present invention will be described more fully with reference to the accompanying drawings.

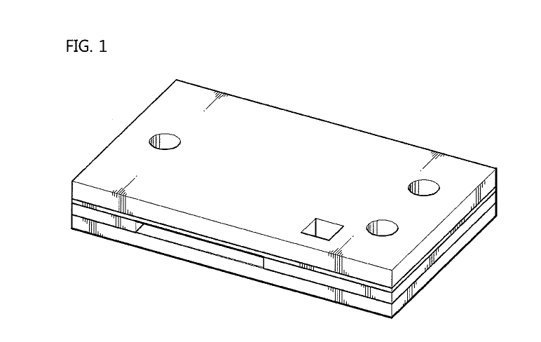

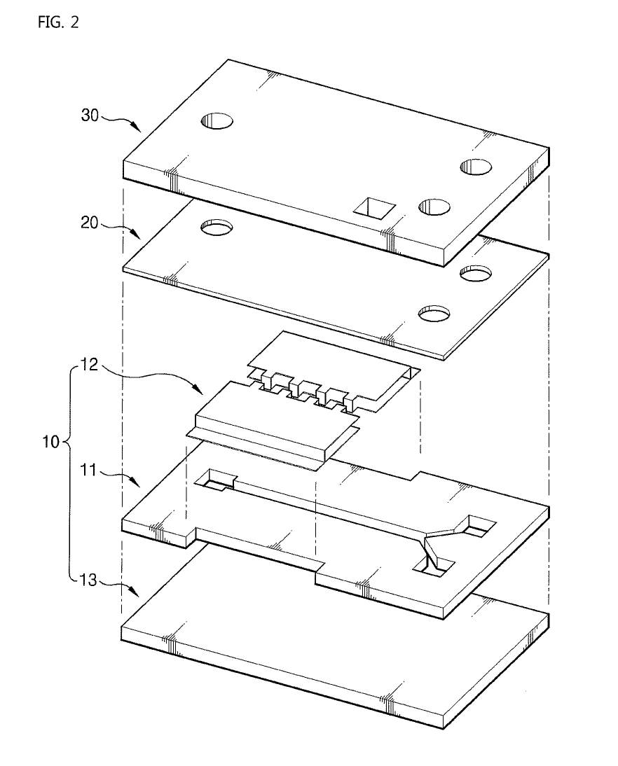

FIG. 1 is a perspective view of a microdevice for fusing cells, according to an embodiment of the present invention, and FIG. 2 is an exploded perspective view of the microdevice. For convenience of illustration, a power supply unit connected between first and second electrodes is not shown, but the power supply unit would have been obvious to one of ordinary skill in the art.

An embodiment of the present invention provides a microdevice for fusing cells, the microdevice including: a microchannel layer 11 including a main microchannel 111 and a plurality of sub-microchannels branched from one end of the main microchannel, wherein an outlet hole 114 is formed at the other end of the main microchannel and a first cell inlet hole 112 and a second cell inlet hole 113 are respectively formed at ends of each of the plurality of sub-microchannels; a plurality of first electrodes 121 formed on one side of the main microchannel; a plurality of second electrodes 122 formed on the other side of the main microchannel and each second electrode facing the each of the first electrodes; a thin film 20 disposed on the microchannel layer and covering the main microchannel; an upper cover 30 including an air inflow passage 31 for connecting a top of the thin film and the outside of the microdevice; and a power supply unit for applying voltage to the plurality of first electrodes and the plurality of second electrodes.

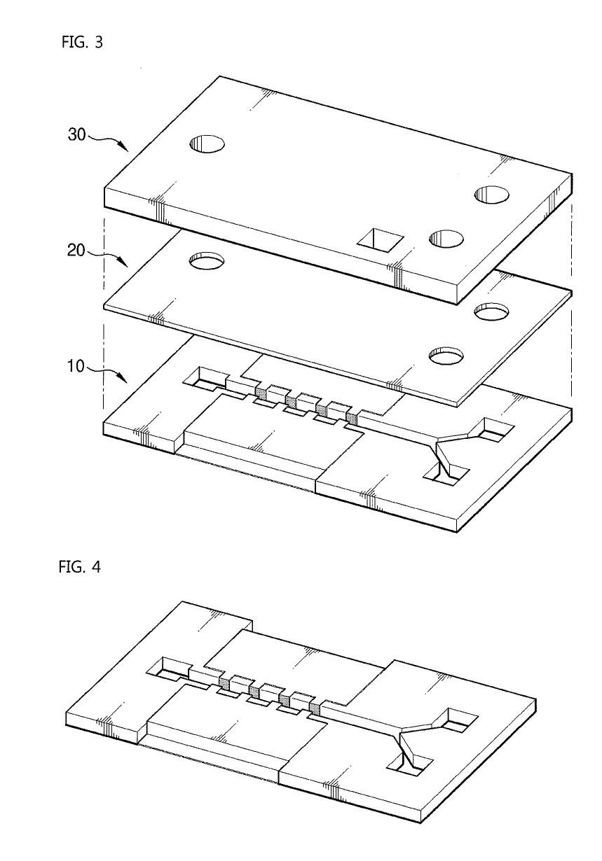

According to the current embodiment of the present invention, the microdevice includes a lower portion 10, a thin film 20, and an upper cover 30 as shown in FIG. 3. The lower portion 10 will now be described in detail.

As shown in FIG. 4, the lower portion includes a microchannel layer 11, a plurality of first electrodes 121 formed on a sidewall of a microchannel, and a plurality of second electrodes 122 respectively facing the first electrodes 121. Also, a substrate 13 may be further disposed below the microchannel layer 11.

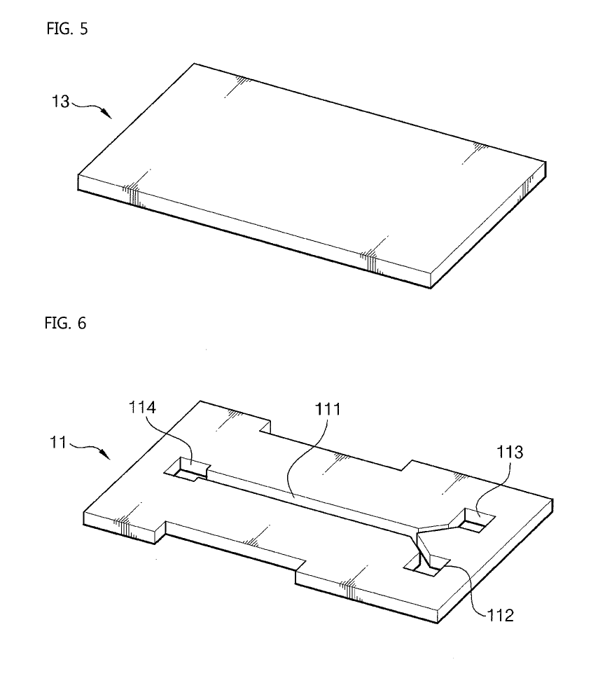

According to an embodiment of the present invention, the microchannel layer 11 may be formed on the substrate 13. The substrate 13 is disposed at the lowest bottom of the microdevice and performs an operation of a supporter as an insulator. A material for forming the substrate 13 is not limited as long as it is an insulating material, and in detail, the material may be silicon, silicon oxide, or glass quartz. A thickness of the substrate 13 is not limited as long as it performs the operation as a supporter, and may be equal to or above 400 μm (refer to FIG. 5).

According to an embodiment of the present invention, the microchannel layer 11 includes a main microchannel 111, and a plurality of sub-microchannels branched from one end of the main microchannel 111. An outlet hole 114 may be formed at another end of the main microchannel 111, and the first cell inlet hole 112 and the second cell inlet hole 113 may be respectively formed at ends of the sub-microchannels as shown in FIG. 6.

The main microchannel 111 and the sub-microchannels are passages where cells flow through. Two types of cells introduced respectively from the first and second cell inlet holes 112 and 113 meet at the main microchannel 111, and the two types of cells are fused in the main microchannel 111. Then, the fused cells are discharged through the outlet hole 114 formed at the other end of the main microchannel 111.

The microchannel layer 11 may be formed of a material that is biocompatible, dysoxidative, noncorrosive, and electric resistive. In detail, Durimide 7510 may be used as the material, but the material is not limited thereto. Alternatively, a photosensitive material may be used.

The first electrodes 121 are formed on one sidewall and the second electrodes 122 facing the first electrodes 121 are formed on the other sidewall of the main microchannel 111 where cells are fused. A voltage is applied to the first and second electrodes 121 and 122 through the power supply unit, and thus two cells in the main microchannel 111 between the first and second electrodes 121 and 122 are fused.

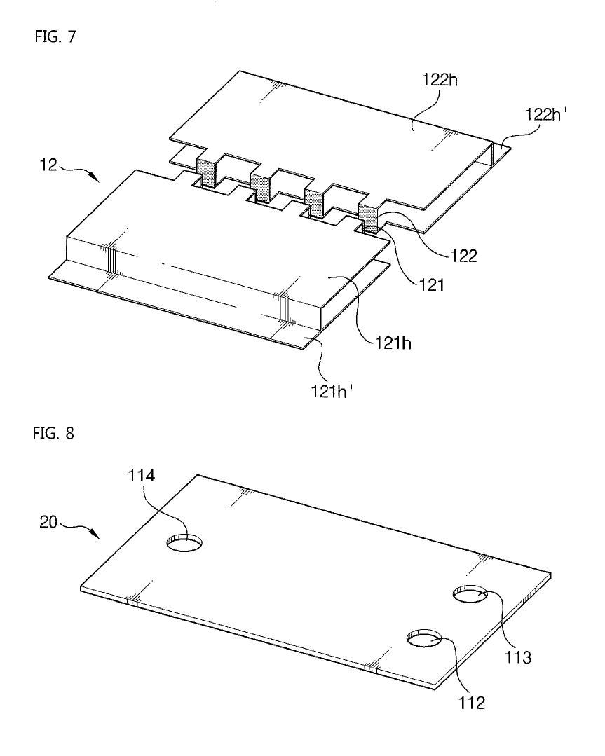

According to an embodiment of the present invention, the first electrodes 121 and the second electrodes 122 are respectively electrically connected to holding pads 121h and 122h having a shape of [symbol] . The holding pads 121h and 122h may be manufactured to have a length and a height corresponding to those of the main microchannel 111, thereby being fit and fixed to a side of the main microchannel 111 to surround all of the bottom, side, and top of the main microchannel 111. FIG. 7 illustrates the first electrodes 121 and the second electrodes 122, which are respectively electrically connected to the holding pads 121h and 122h. As shown in FIG. 7, the holding pads 121h and 122h may respectively include pad shapes 121h′ and 122h′, which receive a predetermined voltage from the power supply unit.

Each of the first or second electrodes 121 or 122 is formed on the sidewall of the main microchannel 111, and may have a height corresponding to a depth of the main microchannel 111 and a width corresponding to 1 to 1.5 times of a diameter of a single cell injected into the main microchannel 111. The first or second electrodes 121 or 122 arranged on the sidewall of the main microchannel 111 may be disposed at an interval of 3 to 4 times of a diameter of a single cell so that two types of cells in the main microchannel 111 are easily fused in an one-to-one manner. Accordingly, a repeated structure of an electrode and a wall of the side of the main microchannel 111 is formed on the side of the main microchannel 111.

A number of electrodes arranged on the side of the main microchannel 111 corresponds to a length of the main microchannel 111, i.e., as the length of the main microchannel 111 increases, the numbers of the first and second electrodes 121 and 122 increase. Accordingly, the lengths of the holding pads 121h and 122h surrounding the main microchannel 111 are also increased.

The holding pads 121h and 122h, the first electrodes 121, and the second electrodes 122 may be formed of a material that is biocompatible, dysoxidative, noncorrosive, and electric conductive. Examples of such a material include gold, platinum, and titanium, but are not limited thereto. Thicknesses of the holding pads 121h and 122h, the first electrodes 121, and the second electrodes 122 may be from 0.2 to 2 μm for excellent electric conductivity, but are not limited thereto.

According to an embodiment of the present invention, the depth of the main microchannel 111 may be from 17 to 30 μm, but is not limited thereto. The width of the main microchannel 111 may be equal to or above a sum of diameters of the first and second cells, and below 1.5 times of the sum of the diameters of the first and second cells. Then length of the main microchannel 111 is proportional to the number of electrodes disposed on the side of the main microchannel 111, and may be a little longer than the disposed electrodes. The sub-microchannels operate as passages where cells introduced from each of the first and second cell inlet holes 112 and 113 flow through. A width of the sub-microchannel may be equal to or above a diameter of a single cell and below 1.5 times of the diameter of the single cell.

A thin film that is flexible, deformable, and covering the main microchannel 111 is disposed on the microchannel layer 11. The thin film is not limited as long as it is flexible and deformable, and in detail, may be a polydimethylsiloxane (PDMS) thin film. A thickness of the thin film may be from 1 to 15 μm. A length and a width of the thin film may be sufficient enough to at least cover the main microchannel 111, and cover the entire microchannel layer 11 at maximum. If the thin film has the length and width covering the entire microchannel layer 11, holes are formed on the thin film at locations corresponding to the outlet hole 114, the first cell inlet hole 112, and the second cell inlet hole 113 of the microchannel layer 11. FIG. 8 illustrates the thin film including the holes. Here, diameters of the holes corresponding to the outlet hole 114, the first cell inlet hole 112, and the second cell inlet hole 113 may be from 1 to 5 mm or 1 to 3 mm, but are not limited thereto.

The upper cover 30 disposed on the thin film includes an air inflow passage 31 connecting the top of the thin film to the outside. According to an embodiment of the present invention, the upper cover 30 may have a thickness from 50 to 400 μm or 70 to 200 μm, and may be formed of PDMS, but is not limited thereto.

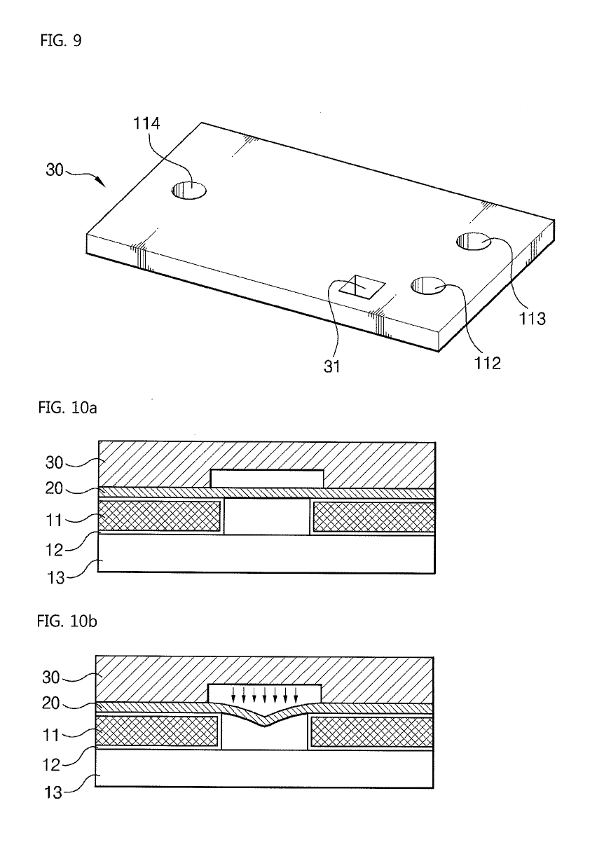

An example of the upper cover 30 is shown in FIG. 9. The upper cover 30 is used to cover the microchannel layer 11 covered by the thin film, and includes holes at locations corresponding to the outlet hole 114, the first cell inlet hole 112, and the second cell inlet hole 113 so that samples easily flow in and out. Here, diameters of the holes corresponding to the outlet hole 114, the first cell inlet hole 112, and the second cell inlet hole 113 may be from 1 to 5 mm or 1 to 3 mm, but are not limited thereto.

A channel having a width wider than that of the main microchannel 111 is formed in the upper cover 30, so that air received from the air inflow passage 31 flows through the channel. A depth of the channel in the upper cover 30 may be from 17 to 30 μm, but is not limited thereto. When the air flows into the upper cover 30, the thin film below the upper cover 30 bends downward according to air pressure, and thus bends toward the main microchannel 111.

An embodiment of the present invention provides a method of fusing cells, the method including: providing the microdevice; bending a thin film toward a main microchannel covered by the thin film by injecting air to a top of the thin film through an air inflow passage of a top cover; injecting first cells and second cells into respective inlet holes, and flowing the first and second cells through a sub-microchannel to the main microchannel; applying an AC voltage between a first electrode and a second electrode such that the injected first and second cells are aligned in the main microchannel according to a dielectrophoresis; performing electroporation on the aligned first and second cells by applying DC pulses between the first electrode and the second electrode; applying a quasi-damping AC voltage between the first electrode and the second electrode such that the electroporated first and second cells are fused by being adjacently disposed to each other according to a dielectrophoresis; relaxing the deformed thin film by releasing the air; and obtaining the fused first and second cells through an outlet hole.

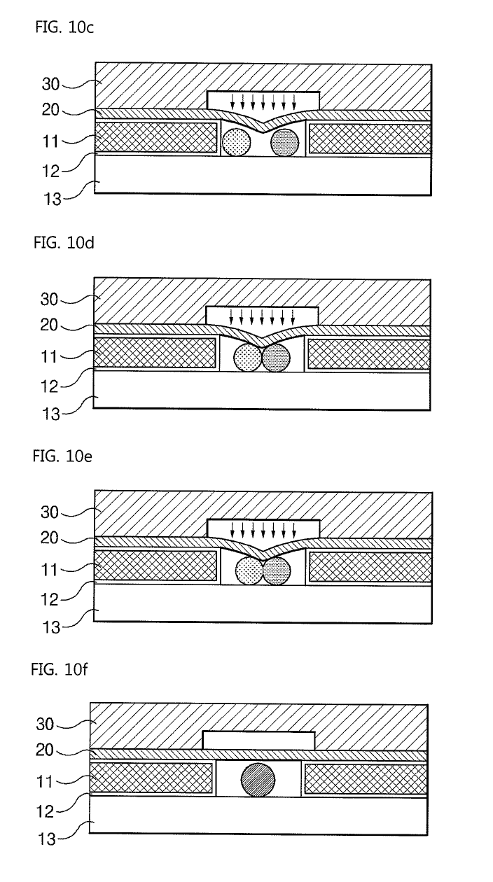

FIGS. 10A through 10F are schematic internal cross-sectional views for describing operations of a microdevice for fusing cells. The operations will now be described with reference to FIGS. 10A through 10F.

First, the microdevice for fusing cells is provided. The microdevice has a cross-section where a thin film covers a main microchannel disposed below the thin film, and an upper cover including a channel wider than the main microchannel is disposed on the thin film, as shown in FIG. 10A.

When air is injected through an air inflow passage of the upper cover, the thin film below the upper cover bends downward according to air pressure, and thus bends toward the main microchannel as shown in FIG. 10B. Accordingly, the inside of the main microchannel is divided into two, and thus substantially two microchannels are generated.

Then, a first cell and a second cell are injected respectively through first and second cell inlet holes, and are flowed through the main microchannel. Here, since the thin film divides the main microchannel into two according to air pressure, and a width of the main microchannel is equal to or above a sum of diameters of the first and second cells, and is below 1.5 times of the sum of the diameters of the first and second cells, the first and second cells are not mixed and flow through the main microchannel each in a line as shown in FIG. 10C.

Then, an AC voltage (amplitude: 2-20V, frequency: 0.2-3 MHz) is applied between first and second electrodes such that the injected first and second cells are aligned in the main microchannel according to dielectrophoresis. Due to the thin film bending toward the main microchannel, a strongest electric field is formed at the center of the main microchannel, and thus the first and second cells are adjacently arranged at the center according to positive dielectrophoresis as shown in FIG. 10D. Next, electroporation is performed on the first and second cells that are adjacently arranged by applying DC pulses (amplitude: 6-50V, duration: 10-500 μs, interval of two pulses: 0.1-10 s, pulses: 1-100) between the first and second electrodes. When the DC pulses are applied, the first and second cells are reversibly electroporated.

Next, a quasi-damping AC voltage (amplitude: 1-2 V, frequency: 0.2-3 MHz, attenuation rate: −0-90%/min) is applied between the first and second cells such that the electroporated first and second cells are adjacently disposed and fused according to dielectrophoresis, as shown in FIG. 10E.

Then, as shown in FIG. 10F, the deformed thin film is relaxed by discharging the injected air, and the fused first and second cells are obtained through an outlet hole. The fused first and second cells may be obtained through the outlet hole by using a syringe pump or electrophoresis, but a method of obtaining the fused first and second cells is not limited thereto.

According to the present invention, the first and second cells may exist between the first and second electrodes each in a line according to the thin film disposed on the microchannel and the air flowing to the thin film, and thus the first and second cells having different traits may be smoothly fused in an one-to-one manner when an electric field is applied between the first and second electrodes.

While the present invention has been particularly shown and described with reference to exemplary embodiments thereof, it will be understood by those of ordinary skill in the art that various changes in form and details may be made therein without departing from the spirit and scope of the present invention as defined by the following claims.

What is claimed is:

1. A microdevice for fusing cells, the microdevice comprising:
a microchannel layer comprising a main microchannel and a plurality of sub-microchannels branched from one end of the main microchannel, wherein an outlet hole is formed at the other end of the main microchannel and a first cell inlet hole and a second cell inlet hole are respectively formed at ends of each of the plurality of sub-microchannels;
a plurality of first electrodes formed on one side of the main microchannel;
a plurality of second electrodes formed on the other side of the main microchannel and each second electrode facing the each of the first electrodes;
a thin film disposed on the microchannel layer and covering the main microchannel;
an upper cover comprising an air inflow passage for connecting a top of the thin film and the outside of the microdevice; and
a power supply unit for applying voltage to the plurality of first electrodes and the plurality of second electrodes.

2. The microdevice of claim 1, wherein each of the plurality of first electrodes and each of the plurality of second electrodes are electrically connected to a holding pad having a shape of [symbol] , wherein the holding pad is fit and fixed to a side of the main microchannel.

3. The microdevice of claim 1, wherein a width of the main microchannel is equal to or above a sum of diameters of a first cell and a second cell, and is below 1.5 times of the sum of the diameters of the first and second cells.

4. The microdevice of claim 1, wherein the thin film is flexible and deformable.

5. The microdevice of claim 1, wherein the thin film is a polydimethylsiloxane (PDMS) thin film.

6. A method of fusing cells, the method comprising:
providing the microdevice of claim 1;
bending a thin film toward a main microchannel covered by the thin film by injecting air to a top of the thin film through an air inflow passage of a top cover;
injecting first cells and second cells into respective inlet holes, and flowing the first and second cells through a sub-microchannel to the main microchannel;
applying an alternating current (AC) voltage between a first electrode and a second electrode such that the injected first and second cells are aligned in the main microchannel according to a dielectrophoresis;
performing electroporation on the aligned first and second cells by applying direct current (DC) pulses between the first electrode and the second electrode;
applying a quasi-damping AC voltage between the first electrode and the second electrode such that the electroporated first and second cells are fused by being adjacently disposed to each other according to a dielectrophoresis;
relaxing the deformed thin film by releasing the air; and
obtaining the fused first and second cells through an outlet hole.
